(19) United States
(12) Patent Application Publication
(10) Pub. No.: US 2015/0027598 A1
(43) Pub. Date: Jan. 29, 2015

(54) CHROMIUM-NICKEL STEEL, MARTENSITIC WIRE AND METHOD FOR PRODUCTION THEREOF

(71) Applicants: Stefan Seng, Essen (DE); Gisbert Klossulitzka, Neuenrade (DE); Osakar Pacher, Graz (AT); Günter Schnabel, Hagen (DE)

(72) Inventors: Stefan Seng, Essen (DE); Gisbert Klossulitzka, Neuenrade (DE); Osakar Pacher, Graz (AT); Günter Schnabel, Hagen (DE)

(21) Appl. No.: 13/807,563

(22) PCT Filed: Jun. 28, 2011
(86) PCT No.: PCT/EP2011/003155

§ 371 Date: Feb. 5, 2013

(30) Foreign Application Priority Data
Jun. 28, 2010 (DE) 10 2010 025 287,5

(51) Int. Cl. C21D 8/06; C21D 6/00; C21D 9/52; C21D 6/02; C21D 1/26; C21D 1/25; C22C 38/58; C22C 38/52; C22C 38/50; C22C 38/48; C22C 38/46; C22C 38/44; C22C 38/42; C22C 38/04; C22C 38/06; C22C 38/00; A61C 7/14; A61L 31/02; C21D 6/04

(52) U.S. Cl. CPC C21D 8/065; C21D 6/04; C21D 6/004; C21D 6/005; C21D 6/007; C21D 6/008; C21D 9/525; C21D 6/02; C21D 1/26; C21D 1/25; C22C 38/58; C22C 38/52; C22C 38/50; C22C 38/48; C22C 38/46; C22C 38/44; C22C 38/42; C22C 38/04; C22C 38/06; C22C 38/001; A61C 7/14; A61L 31/022; C21D 2211/008; C21D 2211/004
USPC 148/578; 148/597; 420/38; 148/310

(57) ABSTRACT

A hardenable chromium-nickel steel, comprising 0.005 to 0.12% carbon, 9 to 17% chromium, 5 to 12% nickel, at most 3% cobalt, 0.5 to 4% molybdenum, 0.25 to 1.0% silicon, 0.5% to 3.0% manganese, 1 to 3% titanium, 0.25 to 1% vanadium, 0.05 to 0.5% niobium, 0.001 to 0.30% nitrogen, at most 0.5% tantalum, 0.001 to 0.030% sulfur, 0.2 to 2.0% copper, at most 0.5% tungsten, at most 1.5% aluminum, 0.0001 to 0.01% boron, and at most 0.035% phosphorus, remainder iron including impurities resulting from smelting, is suitable in particular as a material for producing wire by drawing in the 3-phase region of α-martensite, ε-martensite, and austenite, in conjunction with a heat treatment. The wire can be used for components for instrument construction, surgical needles, valve pins, and dental braces.

The invention relates to a hardenable chromium-nickel steel having a martensitic matrix with carbidic and carbidic/nitridic precipitations and inter-metallic compounds which beside a high tensile strength has a high magnetic saturation and can be bent without significant strain hardening.

Due to their chromium content, chromium steels are corrosion resistant and, depending on their composition, can have an austenitic structure. In this case, they are nonmagnetic and relatively soft and therefore well formable, however, they cannot be hardened and can therefore not be used as material for medical technical devices or components.

In contrast, chromium steels with a predominantly martensitic structure are magnetic; for martensite formation below a critical temperature (Ms-temperature), they require a high cooling rate and/or a strong deformation, depending on their composition. The α-martensite generated thereby which is dependent on the deformation has, as in the case for the normal cooling-dependent α′ martensite, a tetragonally distorted body centered cubic structure, a high hardness and good magnetic properties, however, due to its brittleness it is only poorly deformable. Because of their high hardness and cutting durability, which are due to a high proportion of chromium carbides and carbonitrides, the martensitic chromium steels are suitable as materials for producing blades and knives. As material for producing fine wire these steels are, however, very coarse grained because of the incorporated row-shaped carbides and/or carbonitrides, and not appropriate for producing in particular very thin fine wire, because the carbides and carbonitrides act as breaking nuclei during drawing.

Better deformable than α or α′ martensitic chromium steels are chromium steels with a hexagonal crystal lattice made of ε-martensite which under certain circumstances forms from or in addition to the α-martensite.

Ferritic chromium steels have a body-centered cubic crystal lattice; they cannot be hardened and are not appropriate for producing fine wire with high tensile strength; although they are magnetizable and can be hardened by a heat treatment, they have the disadvantage that their body centered cubic crystal lattice has insufficient solubility for numerous alloy elements. Therefore, embrittling precipitations often form at the grain boundary.

The German patent 602 02 598 T2 discloses a martensitic precipitation hardenable chromium nickel steel with a high strength, tenacity and corrosion resistance for producing cold strip. This steel contains at most 0.03% carbon, 0.5% manganese, 0.5% silicon, 0.04% phosphorous and 0.025% sulfur and 9 to 13% chromium, 7 to 9% nickel, 3 to 6% molybdenum, at most 0.75% copper, 5 to 11% cobalt, at most 1.0% titanium, 1 to 1.5% aluminum and at most 1.0% niobium, 0.01% boron, 0.030% nitrogen and 0.02% oxygen, remainder iron including smelting related impurities.

This steel, particularly due to its high content of cobalt is associated with significant production costs and because of its high contents of molybdenum titanium and aluminum tends to aging.

Further, German patent 692 30 437 T2 describes a precipitation hardenable martensitic chromium nickel steel with 10 to 14% chromium, 7 to 11% nickel, 0.5 to 6% molybdenum, 0.5 to 4% copper, 0.05 to 0.055% aluminum, 0.4 to 1.4% titanium, up to 0.03% carbon and nitrogen, respectively less than 0.05% sulfur and phosphorus, and respectively up to 0.5% manganese and silicon, up to 0.2% tantalum, niobium, vanadium and tungsten, optionally up to 9% cobalt and 0.0001 to 0.1% boron, remainder iron and smelting related impurities.

Due to its low contents of carbon and nitrogen, this steel is free of carbides and carbonitrides; its hardness and strength are due to inter metallic precipitation phases of titanium and aluminum. These precipitations in combination with a relatively high molybdenum content are associated with an embrittlement. The steel is useful as material for producing strip and pipes with a relatively small diameter and small wall thickness.

Further, German patent 693 18 274 T2 describes a martensitic chromium nickel steel with high strength and with respectively between 11.5 and 12% chromium, 9.5 and 10.2% nickel, 1.7 and 5.6% titanium and up to 4.7% molybdenum and optionally between 1.7 and 5.6% tantalum, less than 0.1% carbon, remainder iron including trace elements. This steel contains inter metallic titanium phases and as the case may be also tantalum phases and is therefore subject to the risk of over-ageing.

Further, published PCT application 2006/068 610 A1 describes a precipitation hardenable martensitic chrome steel which contains titanium sulfides, which however is free of manganese sulfides and contains respectively at most 0.07% carbon and 1.5% silicon, 0.2 to 5% manganese, 0.01 to 0.4% sulfur, 10 to 15% chromium, 7 to 14% nickel, 1 to 6% molybdenum, 1 to 3% copper, 0.3 to 2.5% titanium, 0.2 to 1.5% aluminum and at most 0.1% nitrogen, remainder iron. This steel is supposed to ensure an improved machinability and a favorable chip breaking however it poses the risk that its titanium sulfide precipitations can cause structural breaks which prohibit the production of thin wires.

Finally, published PCT application WO 2006/081 401 A1 describes a martensitic stainless steel with a e-Ni3 Ti precipitation phase which contains 8 to 15% chromium, 2 to 15% cobalt, 7 to 14% nickel and up to about 0.7% aluminum, less than about 0.4% copper, 0.5 to 2.5% molybdenum, 0.4 to 0.75% titanium, up to about 5% tungsten and up to about 0.012% carbon, however which requires with regard to the formation of a precipitation phase a forging. This steel is associated with high costs at high cobalt contents and is not appropriate for the drawing of thin wires.

Based on this state of the art, the invention is based on the object to produce a chromium nickel steel which is not only corrosion resistant and can easily be hardened, but also has a high strength and depending on its composition also an excellent bending behavior and a higher magnetic saturation.

This object is solved with a hardenable steel with

0.005 to 0.12% carbon
9 to 17% chromium
5 to 12% nickel
up to 3% cobalt
0.5 to 4% molybdenum,
0.25 to 1% silicon
0.5 to 3% manganese
1 to 3% titanium,
0.25 to 1% vanadium,
0.05 to 0.5% niobium,
up to 0.30% nitrogen,
up to 0.5% tantalum,
up to 0.030% sulfur,
0.2 to 2.0% copper,
up to 0.5% tungsten,
up to 1.5% aluminum, to 0.01% boron,
at most 0.035% phosphorous,
remainder iron including smelting related impurities.

The steel is suitable for producing corrosion resistant wire by multiple drawing and annealing in the 3-phase region a-martensite, e-martensite and austenite at e-martensite proportions of for example up to 30% and a completed wire structure made almost entirely of a-martensite and—due to the composition of the steel—free of ferrite.

Carbon is an austenite former; it promotes and stabilizes the nonmagnetic body-centered cubic crystal lattice and causes carbide precipitations, which lead to an increase in hardness and abrasion resistance. With regard to the desired carbide precipitations in the austenite, the steel should preferably contain at least 0.02% carbon, however not more than 0.12%, because otherwise larger carbides are formed which significantly interfere with the production of fine wire.

Nitrogen serves as nitride former and like carbon, stabilizes the austenitic body centered cubic crystal lattice; it forms nitrides and carbonitrides with the carbide forming elements and in the course of this replaces partially or completely the carbon. Because the proposed steel however is not to contain pure nitride precipitations, the nitrogen content is at least 0.001% and is limited to at most 0.30% and the overall content of carbon and nitrogen should be 0.04 to 0.30%, because in this range carbonitrides advantageously form.

Silicon serves as de-oxidation means. The silicon content however should not exceed 1% because silicon is a ferrite former and therefore lowers the austenite proportion.

Nickel stabilizes the austenitic structure and together with other elements such as titanium, tantalum, niobium, vanadium and copper forms hexagonal precipitations of the type Ni₃Me, which are partially present as mixed crystals and can have lattice defects. Nickel contents below 5% cause the austenitic structure to become unstable with the consequence that the martensite formation occurs prematurely during cooling. On the other hand the stabilization of the austenitic structure is too extensive above 12%. The nickel content is therefore 5 to 12%.

Cobalt also stabilizes the austenite and has a positive effect on the previously mentioned hexagonal precipitations due to its hexagonal crystal lattice. In addition, cobalt increases the saturation magnetization in the magnetic a-martensite and the high temperature strength. In view of the high cobalt price the upper content limit should however be 3%.

Manganese stabilizes the austenite; it therefore shifts the martensite formation to lower temperatures, which is why the manganese content is limited to 3%. Because manganese in combination with nickel, cobalt and chromium also influences the precipitation behavior and solution behavior of the e-martensite and the fine precipitations of the type Ni₃Me, the contents of these elements are preferably adjusted to one another as follows:

(% Ni)+(% Co)+(% Mn)/(% Cr)=0.4 to 2.0%.

The sulfur content is limited to 0.001% to 0.030%, because at higher contents sulfidic precipitations form which have an embrittling effect.

With regard to the corrosion or pitting resistance, in particular in physiological saline solutions, the steel contains 9 to 17% chromium. Chromium in interaction with molybdenum and tungsten reduces pitting, therefore the pitting resistance equivalent should preferably be

(% Cr)+3(% Mo)+(% W)=10 to 30%.

Tungsten together with iron and molybdenum forms mixed carbides and increases the high temperature strength; in addition, it forms precipitations from higher carbides such as for example M₂₃C₆ with favorable solubility. The tungsten content is however, at most 0.5% because higher tungsten contents lead to a deterioration of ductility.

Vanadium, niobium titanium and tantalum form per se carbides and nitrides, however, in the present case because of the high nickel content they form inter-metallic compounds with the nickel of the type Ni₃Me, which compounds form fine precipitations in the martensitic structure and significantly increase the strength of the steel. Because these precipitations in addition have a high solubility in the e-martensite their distribution and/or size can be advantageously influenced by changing the proportions of the martensitic phase (a- and e-martensite) by annealing treatment. The partially very substantial differences in the atomic weight influence the diffusion and the solubility behavior of the Ni₃Me precipitations. Thus it is advantageous when the proportion of nickel to the three lighter elements titanium, vanadium and copper is 0.83 to 8.3.

Aluminum serves as deoxidizing agent; otherwise the oxygen would have an embrittling effect. Oxygen together with nickel and aluminum forms hexagonal precipitations which cause a partial hardening.

Copper, at temperatures below 600° C., together with nickel forms precipitations of the type Ni₃Cu; the copper content is therefore 0.2 to 2.0%.

Subsequent to a solution annealing, the proposed steel can be subjected to a targeted cold deformation by drawing in multiple steps, in each case consisting of multiple individual drawings and a particular heat treatment after each deformation step, in order to eventually establish a a-martensitic structure via a multiphase structure composed of a-martensite, e-martensite and austenite. The invention takes advantage of the phenomenon that the austenite under tensile stresses or during drawing transforms into martensite. This occurs in the course of multiple annealing and drawing steps via the formation of instable e-martensite with decreasing proportion because the instable e-martensite eventually transforms into a-martensite under the influence of the tensile stresses. Insofar, during the multistep cold drawing, the e-martensite represents merely an intermediate stage of the structure, until the structure is almost entirely a-martensitic. After the deformation and the annealing, almost the entire proportion of the austenite is transformed thereby withdrawing the basis for the formation of e-martensite. Until then, however, the formation of e-martensite influences the properties of the basic structure (matrix) due to interactions with the precipitations with the result of a homogenous distribution of the precipitations with an extremely low particle size and low tendency to agglomerate.

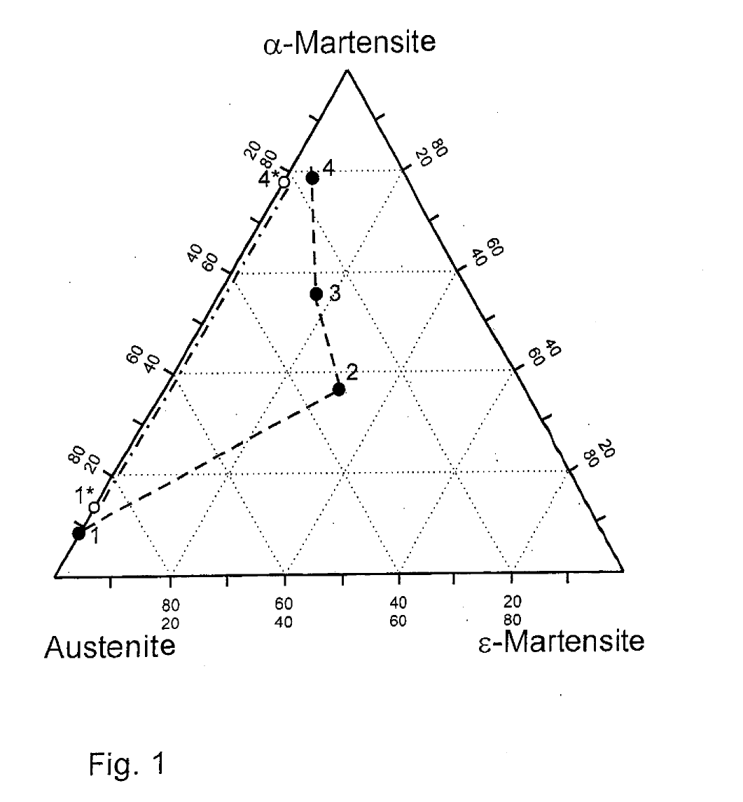

Starting point is the solution annealing or austenizing annealing, namely at 700 to 1100° C. with a holding time of 30 to 60 min, which is targeted toward attaining an austenitic basic structure with hard precipitations of the type MC and M(C, N) with vanadium, titanium, niobium as Me, as well as the type M₂₃C₆ with chromium, iron and molybdenum as M as indicated in FIG. 1 with the point 1.

The austenizing annealing or solution annealing is followed by a cold deformation in multiple steps by means of hard metal drawing stones. The cold drawing is associated with a significant strain hardening depending on the cross section reduction of the wire, which strain hardening necessitates an annealing of the wire in order to enable a further cold deformation and in this way initially promote the formation of hexagonal e-martensite. At the same time, the annealing has the goal to retain dissolved elements (cf. Table I) in solution if possible and to deform the wire in the three phase region a-martensite, e-martensite and austenite, as schematically indicated in the three phase diagram of FIG. 1 by the points 2, 3 and 4 for a three step deformation in each case with an intermediate annealing and a final annealing. The points 1, 2, 3 an 4 indicate in the stepwise drawing and annealing, illustrated by the hatched line, the initial increase of the proportion of e-martensite up to point 2 and the further structural change in the 3-phase region with an increase of the proportion of a-martensite and a decrease of the proportion of e-martensite up to point 4. The hatched line with the points 1*, 4* on the other hand shows the transformation of the austenite into a-martensite without the “detour” according to the invention via an e-martensite formation for typical steels which are not according to the invention.

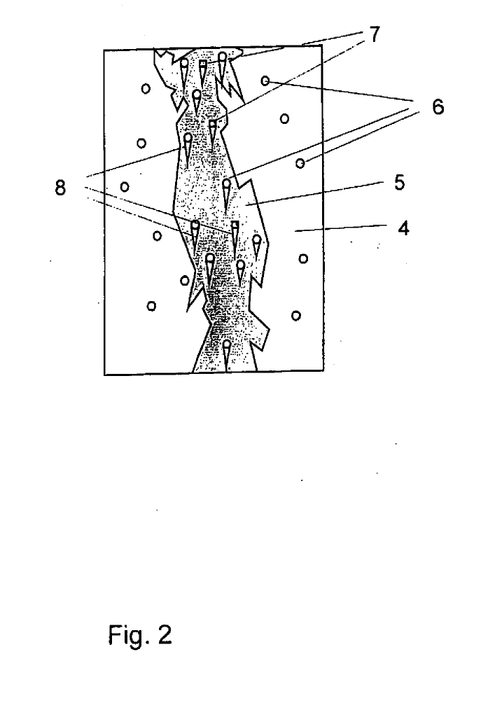

During cold drawing a significant stretching of the wire occurs as a result of the cross section decrease, which is associated by structural changes, as it is schematically shown in the FIGS. 2 to 4.

Under the influence of the cross section decrease and the stretching during drawing a-martensite is locally formed from the austenite at simultaneous increase of the strength of the steel, associated with magnetizability due to the a-martensite. Further, during cold deformation, due to the hard and poorly soluble carbide and/or carbonitride precipitations 6 incorporated in the austenite 4 as well as behind hard yet partially soluble carbides 7 such as M₂₃C₆ which beside chromium, molybdenum and tungsten can also contain iron as M, tip shaped austenitic drawing shadows form with roughly twice to five times the length of the particle width of the primary precipitations incorporated in the structure or carbides and/or carbonitrides, preferably with a grain size of 1 to 4 μm. A prerequisite for this are 0.005 to 0.12% carbon and 0.01 to 0.30% nitrogen.

Because in the region of the drawing shadows 8 no significant deformation or longitudinal stretching occurs, the drawing shadow 8 retains its original austenitic structure. It is however, surrounded by the forming a-martensite 5 with hard carbonitritic precipitations 6. However there is a risk that with increasing number of drawing shadows or their dense distribution in the structure, the stability of the forming martensite decreases significantly. This may be due to the fact that with the number of the drawing shadows their interface with the martensite, i.e., the interface martensite austenite also increases significantly, and as a result the interface energy and with this the energy content of the martensite in which the drawing shadows are incorporated significantly increases as well.

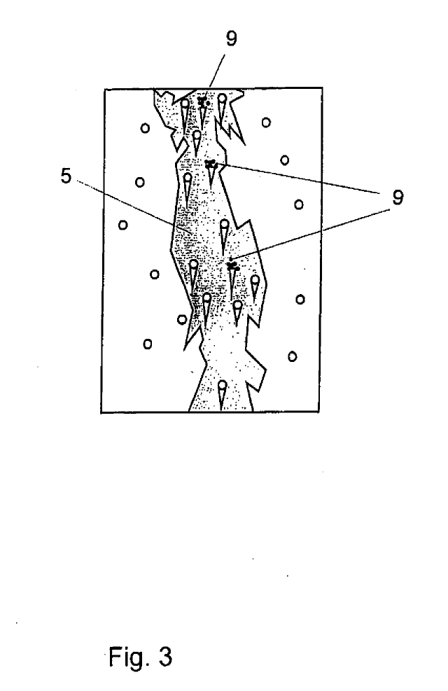

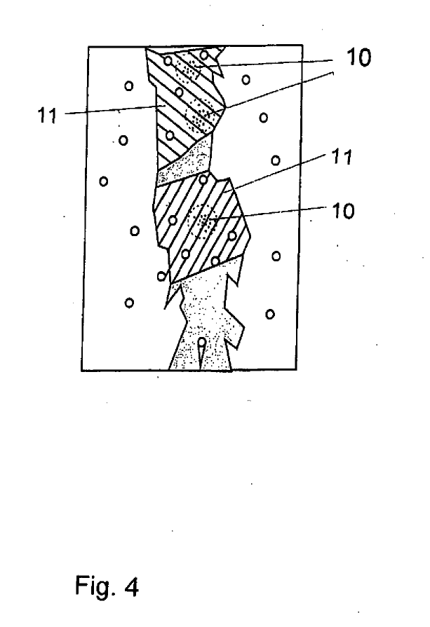

In the stepwise longtime annealing following the austenizing annealing with a duration of three to twelve hours at 650 to 850° C. (WB1), the structure further changes, as can be seen from the schematic representation of the structure of the FIGS. 3 and 4.

In such an annealing, essentially only tensions and offsets are decreased during the first hour which enables a better deformation of the wire. In the long-term annealing according to the invention of more than two hours however, hexagonal martensite forms in a matrix of austenite and a-martensite, which can be detected by etching with potassium meta-bisulfate and by magnetically measuring the quantities.

Tests have shown that the three phases are formed in two stages. In the first stage, certain precipitations such as M₂₃C₆ carbides 7 in FIG. 2 are partially dissolved or re-dissolved; thereby releasing dissolved and then released atoms from partially soluble precipitations 9. As a result, the energy content of the a-martensite further increases beyond the already significant level caused by the high interface energy of the drawing shadows 8 until instability. In this time-dependent dissolving or re-dissolving of fine precipitations 10 (FIG. 4), the structure eventually adopts a state in which after an intermediate annealing in the second step of the annealing, the a-martensite 5 exceeds the limit of its stability and locally transforms into the energy rich hexagonal e-martensite 11 according to FIG. 4, which provides a better solubility for many elements. The formation of the e-martensite is determined by a complex synergetic interaction of different variables, for example the drawing shadows which, owing to their higher interface energy presumably function as nuclei for the fine precipitations 10 such as Ni₂₃M.

Of particular importance in this context is that the precipitation phases of the type Ni₃M with titanium, vanadium, niobium, tantalum, aluminum and copper as M all have a hexagonal crystal structure and at least for titanium vanadium and copper have a better solubility in the e-martensite. This results in a better diffusion or distribution in the structure and a facilitation of the microcrystal formation (Table I). This and the fact that hexagonal martensite forms from the tetragonal martensite is an important feature of the invention.

In the multistep longtime annealing of the magnetizable a-martensite thus a three-phase structure of austenite, a-martensite and e-martensite initially forms which is followed by further drawing and longtime annealing steps with the goal to facilitate the formation of e-martensite at a decreasing austenite proportion according to the points 3, 4 in FIG. 1, which becomes more difficult with decreasing austenite proportion. For the previously mentioned hexagonal precipitations, the hexagonal crystal lattice of the e-martensite should be advantageous which promotes the mixed crystal formation (Table I). In the last drawing of the wire, a-martensite forms again and as a result the magnetic saturation increases.

Insofar, the a-martensite is of paramount importance, however, it is also transformed and deformed multiple times during further drawing and annealing at progressive formation and transformation of e-martensite, with the consequence that the properties of the wire are improved owing to a better and more even and finer distribution of the particularly small sized precipitations or particles. This not only results in a soft-annealed and with this also cold deformable wire, but also in particular in a temporary continuous increase of the proportion of e-martensite in the structure.

The wire can have an increased surface roughness after the multistep or three step annealing as a result of the structural change which is associated with a change in volume. In order to remove this roughness, the wire can be drawn once more for smoothening.

The final drawing can be followed by a 30 minute to one hour tempering at 350 to 550° C. as well as alternatively or in addition a low temperature treatment at temperatures below −12° C. for 20 to 40 minutes. This can be followed by a tempering of the same duration at 250 to 400° C., which is optionally followed by a tempering of the same duration at 450 to 550° C.

The wire has in each case a tensile strength of greater than 2000 N/mm² and a magnetic saturation of 200 to 235 Gcm³/g. The high saturation magnetization is a reliable indication for a correspondingly high proportion of a-martensite in the structure, because the steel does not contain ferrite in the structure owing to its composition, and e-martensite is nonmagnetic.

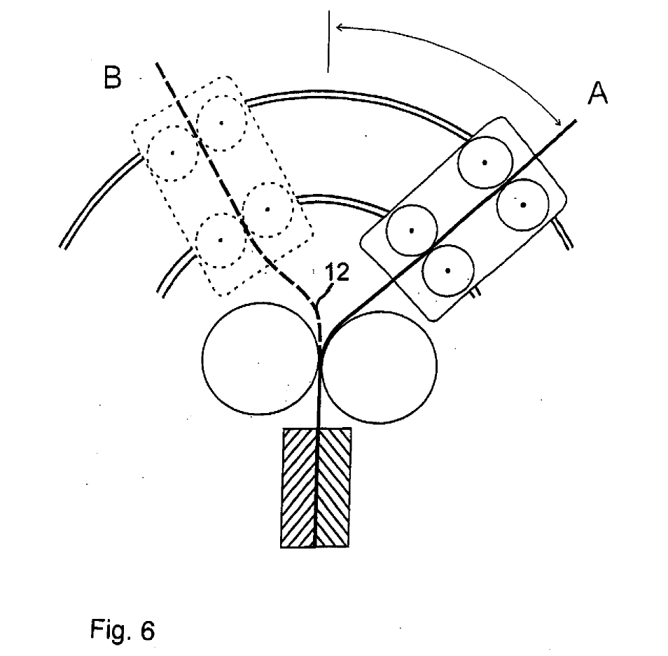

The wire has a maximal diameter of 1 mm, preferably at not more than 0.8 mm, a high strength and elasticity: it does not show any bulges or waves 12 after a bending and a subsequent backward bending by a total of 90°, as is the case for the conventional wire according to FIG. 6 after a forward and backward bending as a result of bending strain hardening. Therefore, it is in particular suitable for producing surgical needles.

### Test Results

In Table II, eight experimental steels are listed of which the steels L1 to L4 are according to the invention and the steels L5 to L8 are reference steels whose differences in composition are in each case in bold print. The sulfur content of the samples does not exceed very narrow limits; it is within the range of 0.003 to 0.008%.

The samples of the test steels were produced at laboratory scale and forged into rods, rolled out into wire with a diameter of about 5.5 mm and annealed 35 min. at 1050° C. The annealed wire was then pickled and drawn to a diameter of 0.8 mm in three drawing steps by using coated, hard metal drawing stones. Between every two drawing steps, the samples were annealed (cf. table III). Then, the respective structure was examined microscopically after an etching with potassium pyrosulfite solution and the magnetic saturation analyzed with a Sigmameter testing device of the company Setarem, Caluier, France. This measurement is conclusive with regard to the structural proportion of the e-martensite as the latter is nonmagnetic in contrast to a-martensite and the mentioned etching stains the austenite and the two martensite phases differently.

The test results for the samples 1 to 15 according to the invention and the samples 16 to 23 of conventional steels are summarized in Table III. After the first annealing, the data for the test steels 1 to 11 according to the invention show an e-martensite proportion of at least 24% which in further drawing and annealing steps almost entirely disappears associated with a decrease in austenite (FIG. 1). A possible residual content of austenite and e-martensite can be almost completely removed with a low temperature treatment. In contrast, in the steels of the tests 12 to 15 which according to the analysis are also according to the invention, the proportion of e-martensite was too low owing to the short annealing time of only 0.5 to 1 hour at a still good tensile strength. In addition to this, the martensite proportions for all drawing steps 2, 3 and 4 are shown by way of example in the phase diagram of FIG. 1. This reveals how the proportion and the ratio of the phase proportions, in particular the ones of e-martensite, change from one drawing step to another or from one annealing step to another. These are structural changes which do not occur in steels which are not according to the invention or the reference steel of FIG. 1 with the points 1*, 4*.

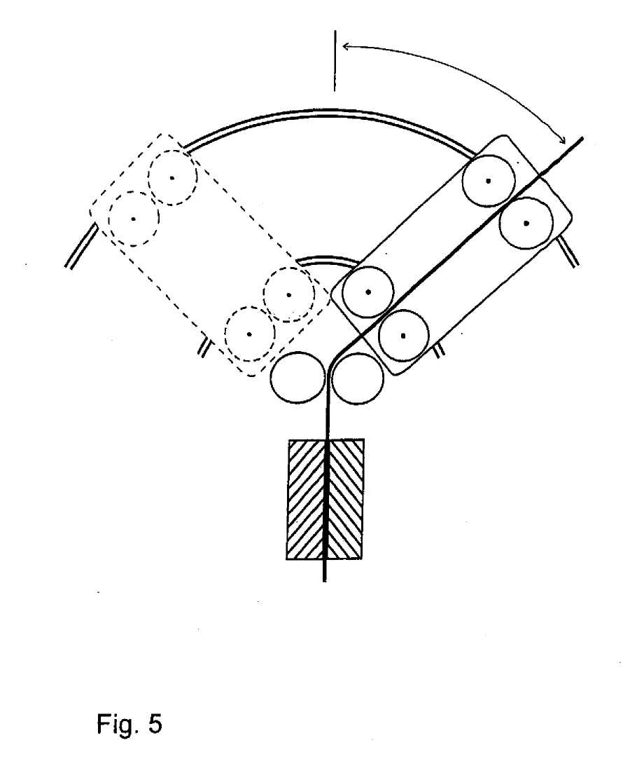

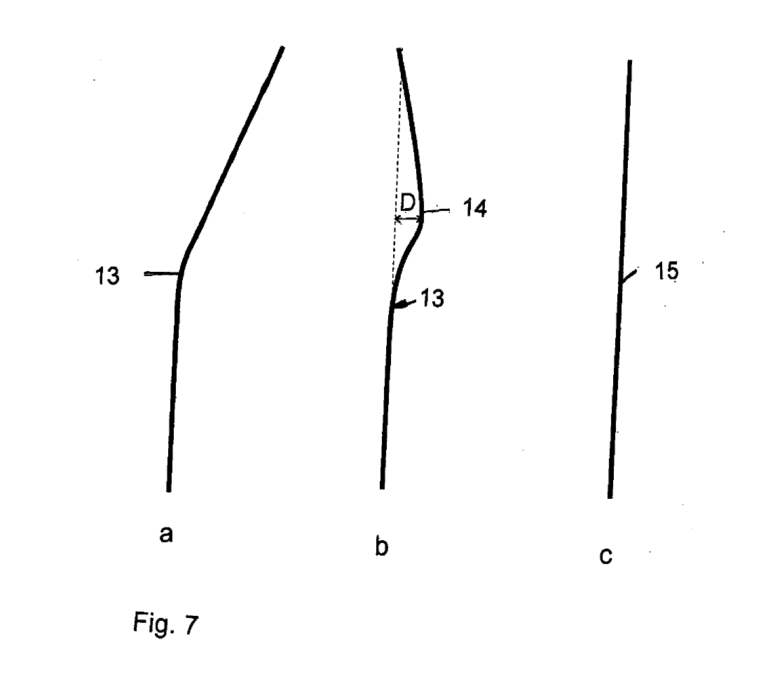

In order to determine the change of the strength in a to and fro bending test, all samples were subjected to a bending of 90° from A to B in the device shown in FIGS. 5, 6. In this test, a lasting bending 13 forms in the region of the maximal bending curvature 12 according to FIG. 6, 7 (FIG. 7a). In the case of a backward bending, two results are possible. In one case, i.e., in the sample according to FIG. 7b, the hardening of the wire leads in the backward bending to the formation of a locally offset bending deformation 14 with the consequence that the first bending 13 remains and a second opposite bending at 14 is added, so that overall a bulge of a height D is formed. However, at an only low bending-strain hardening as in the case of a wire according to the invention, the backward bending can occur at the same point 13 of the wire at which the initial bending deformation had occurred. In this case, a bulge-free wire 15 with D=0 (FIG. 7) results. By means of the described test, the tendency of the wire for bending-strain hardening can therefore be determined by way of measuring the bulge height D. For the tests 1 to 11 according to the invention on one hand, as well as 12 to 15 on the other hand and the tests 16 to 23 with the reference steels 25 to 28, the corresponding data for the bulge height D are summarized in Table III.

Overall, the data of Table III show that the steels L1-L4 according to the invention have a particularly high tensile strength of 2000 N/mm² (Column 9), which is in particular due to a high proportion of hexagonal e-martensite after the first annealing step (column 6) in the heat treated and deformed structure (tests 1 to 12 and 14). Further, the test results show the significant influence of the content limits, such as for example the reference steel L5 in connection with the tests 16 to 18 in a steel with too low carbon content and as a result a deficit of primary carbides. Even after a four hour annealing, no e-martensite forms which is presumably due to the fact that the absence of primary carbides does not allow the formation of drawing shadows, with the consequence that the formation of e-martensite is suppressed. However, when the carbon content exceeds the maximal content as in the steel L8 with 0.30%, coarse carbide precipitations form which lead to a premature fracture, i.e., in the first drawing step.

The tests 19 and 21 to 23 also demonstrate the influence of the composition of the steel during drawing of fine wire. The data show that oversaturated precipitations without the tendency for the formation of e-martensite (column 6) cause a poor bending behavior. This exemplifies the great importance of the three-phase region according to the invention. Thus, the magnetic saturation above the threshold value of 200 Gcm³/g indicates an optimal magnetic martensite phase such as for example in the case of the tests 7 and 8. At saturation values below the previously mentioned threshold value—as in the case of test 20 which is not according to the invention—the test results in contrast are significantly poorer.

Overall, it is thus shown that the multiply formed transformed a-martensite which forms from e-martensite contains a finer and more even distribution of fine precipitations of the type Ni₃Me and thus causes the significantly improved ability and the better bending properties. In the case of a steel analysis according to the invention, the formation of precipitations of the type Ni₃Me is ensured the properties of the wire can thus be optimized by a combination of cold deformation and heat treatment.

TABLE I

| | | Solution temperature at annealing | |
|---|---|---|---|
| | Crystal | 1 hour | 4 hours |
| Precipitations | lattice | ° C. | |
| Ti (C, N) | Cubic | | >1100 |
| TiC | Cubic | | >1100 |
| NbC | Cubic | | >1100 |
| V(C, N) | Cubic | | >1000 |
| Cr₂₃C₆ | Cubic | >800 | >550 |
| Ni₃Ti | Hexagonal | >850 | >600 |
| Ni₃V | Hexagonal | >850 | >600 |
| Ni₃Nb | Hexagonal | >850 | >650 |
| N₃Ta | Hexagonal | >900 | >650 |
| Ni₃Cu | Hexagonal | >600 | >450 |
| Ni₃ (Ti, V, Nb, Ta) | Hexagonal | — | ca. 600 |
| mixed crystal | | | |

TABLE II

| Steel | C % | N % | C + N % | Cr % | Ni % | Co % | Mo % | Mn % | Ti % | V % | Nb % | Ta % | Cu % | W % | Al % |
|---|---|---|---|---|---|---|---|---|---|---|---|---|---|---|---|
| L1 | 0.04 | 0.04 | 0.08 | 9.5 | 7.2 | Traces | 0.55 | 2.12 | 1.25 | 0.90 | 0.08 | 0.06 | 0.35 | 0.25 | 1.25 |
| L2 | 0.03 | 0.02 | 0.05 | 10.0 | 8.5 | Traces | 1.95 | 1.25 | 1.42 | 0.66 | 0.10 | 0.21 | 0.76 | Traces | 0.45 |
| L3 | 0.10 | 0.08 | 0.18 | 9.8 | 8.2 | 1.2 | 1.80 | 0.90 | 1.95 | 0.32 | 0.30 | Traces | 1.75 | 0.15 | 0.28 |
| L4 | 0.09 | 0.01 | 0.10 | 14.5 | 11.0 | 0.5 | 3.55 | 1.90 | 2.00 | 0.28 | 0.35 | 0.12 | 1.02 | 0.35 | 0.15 |
| L5 | 0.005 | 0.025 | 0.03 | 18.0 | 4.5 | 0.1 | 0.10 | 0.75 | 0.25 | 0.12 | 0.02 | Traces | 0.12 | Traces | Traces |
| L6 | 0.002 | 0.004 | 0.06 | 8.0 | 12.2 | 2.1 | 0.08 | 2.40 | 0.20 | 0.18 | 0.65 | 0.15 | 0.66 | Traces | 0.80 |
| L7 | 0.05 | 0.30 | 0.35 | 7.0 | 13.7 | Traces | 4.20 | 2.90 | 3.80 | 2.40 | 0.52 | 0.25 | 3.20 | 0.10 | 0.95 |
| L8 | 0.08 | 0.03 | 0.38 | 7.5 | 12.6 | — | 4.80 | 3.20 | 0.90 | 0.02 | 0.12 | 0.12 | 0.50 | 0.05 | 1.75 |

TABLE III

| | | Assessment | Annealing | | Proportion in % | Cooling | Tempering | Tensile strength | Magn. | Bulge |
|---|---|---|---|---|---|---|---|---|---|---|
| Test No.: | Alloy | wire | Duration/h | T/° C. | ε martensite | T <12° C. | T/° C. | N/mm² | saturation | D/mm |
| 1 | L1 | Good | 4 | 850 | 24 | — | 520 | 2200 | 221.0 | 0.3 |
| 2 | L1 | Good | 5 | 850 | 24 | — | 520 | 2340 | 223.4 | 0.4 |
| 3 | L1 | Good | 5 | 750 | 25 | — | 520 | 2360 | 223.8 | 0.2 |
| 4 | L1 | Good | 6 | 850 | 24 | — | 520 | 2350 | 223.5 | 0.2 |
| 5 | L2 | Good | 4 | 850 | 25 | — | 520 | 2480 | 224.6 | 0.2 |
| 6 | L2 | Good | 4 | 750 | 27 | — | 520 | 2500 | 225.4 | 0.3 |
| 7 | L2 | Good | 6 | 750 | 31 | −196 | 450 | 2800 | 230.5 | 0.4 |
| 8 | L3 | Good | 4 | 750 | 29 | — | 450 | 2750 | 229.5 | 0.3 |
| 9 | L3 | Good | 6 | 750 | 30 | −196 | 450 | 2820 | 229.6 | 0.4 |
| 10 | L4 | Good | 6 | 800 | 26 | — | 450 | 2460 | 224.2 | 0.2 |
| 11 | L4 | Good | 6 | 800 | 26. | — | 450 | 2450 | 224.8 | 0.1 |
| 12 | L1 | — | 0.5 | 850 | n.n. | — | 450 | 2050 | 222.4 | 1.2 |
| 13 | L2 | — | 0.5 | 850 | n.n. | — | 500 | 1950 | 219.6 | 1.1 |
| 14 | L3 | — | 1 | 850 | 5 | — | 500 | 2000 | 217.8 | 1.0 |
| 15 | L4 | — | 1 | 850 | 5 | — | 500 | 1980 | 206.5 | 1.0 |
| 16 | L5 | Poor | 0.5 | 750 | n.n. | — | 500 | 1970 | 196.3 | 2.5 |
| 17 | L5 | Poor | 4 | 700 | n.n. | — | 500 | 1980 | 197.4 | 2.2 |
| 18 | L5 | Poor | 6 | 700 | n.n. | — | 500 | 1970 | 195.0 | 2.0 |
| 19 | L6 | Break, Z3 | 0.5 | 750 | n.n. | — | 520 | — | 169.5 | — |
| 20 | L6 | Poor | 4 | 700 | n.n. | — | 520 | 1710 | 186.5 | 2.6 |
| 21 | L7 | Break, Z2 | 1 | 850 | n.n. | — | — | — | 112.0 | — |
| 22 | L7 | Break, Z3 | 4 | 800 | n.n. | — | — | — | 164.0 | — |
| 23 | L8 | Break, Z1 | — | — | — | — | — | — | 126.5 | — |

Notes:
n.n.: not detectable
* proportion ε-martensite after the 1.annealing Break,
Zi: wire break in the drawing step . . .

TABLE IV

| | |
|---|---|
| Duration (h) | Temperature/° C. |

Long term annealing: WB1

| | |
|---|---|
| 3 to 12 | 650 to 850 |

Tempering: WB2

| | |
|---|---|
| 0.5 to 1.0 | 350 to 550 |

Tempering after subzero cooling: WB3

| | |
|---|---|
| 0.5 to 1.0 | 250 to 400 |

Tempering after subzero cooling: WB4

| | |
|---|---|
| 0.5 to 1.0 | 250 to 400 |
| 0.5 to 1.0 | 450 to 550 |

What is claimed is:

1.-15. (canceled)

16. A hardenable chromium-nickel steel comprising, in weight percent:
0.005 to 0.12% carbon
9 to 17% chromium
5 to 12% nickel
up to 3% cobalt
0.5 to 4% molybdenum,
0.25 to 1% silicon
0.5 to 3% manganese
1 to 3% titanium,
0.25 to 1% vanadium,
0.05 to 0.5% niobium,
0.001 to 0.30% nitrogen,
up to 0.5% tantalum,
0.001 to 0.030% sulfur,
0.2 to 2.0% copper,
up to 0.5% tungsten,
up to 1.5% aluminum,
0.0001 to 0.01% boron,
at most 0.035% phosphorous,
remainder iron including smelting related impurities.

17. The chromium-nickel steel of claim 16, wherein the carbon and nitrogen contents satisfy the following condition:
(% C)+(% N)≦0.3 to 0.04%.

18. The chromium-nickel steel of claim 16, wherein the niobium and tantalum contents satisfy the following condition:
(% Nb)+(% Ta)=0.05 to 0.5%

19. The chromium-nickel steel of claim 16, wherein the chromium, molybdenum and tungsten contents satisfy the following condition:
(% Cr)+3(% Mo)+(% W)=11 to 30%.

20. The chromium-nickel steel of claim 16, wherein the nickel, titanium, vanadium and copper contents satisfy the following condition:
(% Ni)/(% Ti)+(V)+(% Cu)=0.83 to 8.3%.

21. The chromium-nickel steel of claim 16, wherein the nickel, cobalt, manganese and carbon contents satisfy the condition:
(% Ni)+(% Co)+(% Mn)/(% Cr)=0.4 to 2.0%.

22. A method for producing a wire by hot and cold rolling the chromium-nickel steel of claim 16, comprising the steps of:
solution annealing the chromium-nickel steel for maximally 60 min at 750 to 1100° C.;
cold drawing the chromium-nickel steel into the wire in multiple steps so as to establish an a martensitic structure in the wire; and
cold drawing the chromium-nickel steel in individual drawing steps into the wire with intermittent annealing of the wire between every two of the drawing steps so as to establish an a-martensitic structure in the wire.

23. The method of claim 22, further comprising cooling the chromium nickel steel to room temperature after the intermittent annealing, wherein the intermittent annealing is performed for 2 to 12 h at 650 to 850° C.

24. The method of claim 23, further comprising tempering the wire for 30 to 60 min at 350 to 550° C.

25. The method of claim 23, further comprising tempering the wire for 20 to 40 min at below −12° C.

26. The method of claim 23, further comprising tempering the wire for 0.5 to 1 h at 250 to 400° C.

27. The method of claim 26, wherein the steel is aged for 0.5 to 1 h at 280 to 400° C.

28. The method of claim 27, further comprising subjecting the wire to a final tempering for 0.5 to 1 h at 450 to 550° C.

29. A method of using a wire made of the chromium-nickel steel of claim 16 and having an essentially a-martensitic structure, for producing items which require a high strength of at least 2000 N/mm² and a magnetic saturation of 200 to 235 Gcm³/g

30. The method of claim 29, wherein the wire is used as material for producing one of surgical needles, valve pins and dental brackets.
